(19) United States
(12) Patent Application Publication
(10) Pub. No.: US 2004/0013711 A1
(43) Pub. Date: Jan. 22, 2004

(54) Adhesive patch device for preventing inner-thigh skin irritation

(76) Inventor: Sylvia Loretta Lamb, Plano, TX (US)

Correspondence Address: SYLVIA L. LAMB, 3909 DENHAM WAY, PLANO, TX, 75023, US

(21) Appl. No.: 10/213,543

(22) Filed: Jul. 16, 2002

(51) Int. Cl. A61K 9/70; A61F 13/00

(52) U.S. Cl. 424/443

(57) ABSTRACT

What is new about in the nature to which my invention pertains is that is that it is specifically designed for providing comfort to skin irritations on the inner-thigh region. Although many people are bothered by skin irritations and discomfort in the inner thigh regions, where thighs may tend to rub, create friction, sweat, or stick together, often resulting in rash, chaffing, and/or irritation, there is to date no device that specifically answers to this, until now. Accordingly, my invention will provide relief by helping to reduce friction in the inner-thigh region. My invention will help prevent rashes, burns, irritation, and discomfort to the area where the device is applied by preventing direct skin contact and displacing any friction to the patch itself.

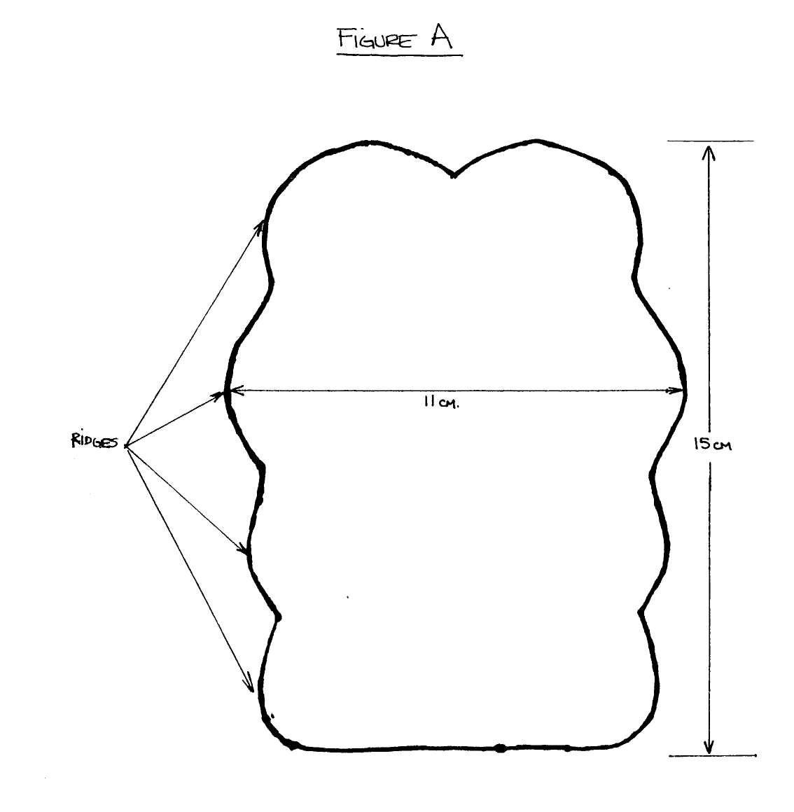

### B. BACKGROUND

### FIELD OF INVENTION

This invention relates to health and beauty, specifically providing comfort to skin irritations on the inner-thigh region.

### C. SUMMARY

Many people are often bothered by skin irritations and discomfort in the inner thigh regions, where thighs may tend to rub, create friction, sweat, or stick together. Often the results turn into rash, chaffing, and/or irritation. Those who often suffer from these types of symptoms are people who are overweight, have sensitive skin, are physically active, or just want to be comfortable.

### D. OBJECTS AND ADVANTAGES

Accordingly, several objects and advantages of my invention are the comfort that the thigh patch will provide by helping reduce friction in the inner-thigh region. The patch will help prevent rashes, burns, irritation, and discomfort to the area where it is applied by preventing direct skin contact and displacing any friction to the patch itself. This product will be especially helpful in preventing pain and discomfort to those who are overweight, active, or simply experiencing skin discomforts associated with the inner thighs.

Further objects and advantages of my invention will become apparent from a consideration of the drawings and ensuing description.

### E. DESCRIPTION OF DRAWINGS

FIG. A is a frontal view of my invention in detail of the portions indicated to express approximate shape as well as height and width of the thigh patch.

### F. DESCRIPTION OF INVENTION

The patch will be made of a bandage-like material that will have an adhesive on one side that will firmly and effectively adhere to the skin. The size and shape of the patch will vary depending on the size of one's thigh contact area. The patch will also be available in several skin tone colors.

1. I claim that my “Adhesive Patch Device for Preventing Inner-thigh Skin Irritation” relating to health and beauty, is specifically designed for providing comfort to skin irritations on the inner-thigh region. Many people are often bothered by skin irritations and discomfort in the inner thigh regions, where thighs may tend to rub, create friction, sweat, or stick together. Often the results turn into rash, chaffing, and/or irritation. Those who often suffer from these types of symptoms are people who are overweight, have sensitive skin, are physically active, or just want to be comfortable.
Accordingly, my invention will provide relief by helping to reduce friction in the inner-thigh region. The patch will help prevent rashes, burns, irritation, and discomfort to the area where it is applied by preventing direct skin contact and displacing any friction to the patch itself. The patch will be made of a bandage-like material that will have an adhesive on one side that will firmly and effectively adhere to the skin. Although the size of the patch will vary depending on the size of the individuals thigh contact area, all patches will be shaped to consist of sets of ridges along the outside cut. These ridges help to effectively insure that the patch remains applied. The patch will be available in several skin tone colors.
